(19) United States
(12) Patent Application Publication
(10) Pub. No.: US 2016/0095994 A1
(43) Pub. Date: Apr. 7, 2016

(54) Hypoxic Breathing Apparatus and Method

(71) Applicant: Third Wind, LLC, Edina, MN (US)

(72) Inventors: Jarrod D. Currin, New York, NY (US); Byron L. Gilman, Edina, MN (US); Mark W. Kroll, Orono, MN (US)

(73) Assignee: THIRD WIND, LLC, Edina, MN (US)

(21) Appl. No.: 14/504,086

(22) Filed: Oct. 1, 2014

(51) Int. Cl. A61M 16/00; A61M 16/12; A61M 16/08; A61M 16/20; A61M 16/22; A61M 16/06

(52) U.S. Cl. CPC A61M 16/0045; A61M 16/22; A61M 16/0078; A61M 16/06; A61M 16/0875; A61M 16/208; A61M 16/0891; A61M 16/201; A61M 16/0051; A61M 16/125; A61M 2205/125; A61M 2205/3569; A61M 2205/3592; A61M 2230/432; A61M 2230/435; A61M 2205/3306; A61M 2230/205; A61M 2230/04; A61M 2230/63; A61M 2230/42; A61M 2205/3303; A61M 2202/0208; A61M 2202/0225; A61M 2202/0266; A61M 2205/50

(57) ABSTRACT

A breathing apparatus includes a breathing mask, hose, and first housing in fluid communication with the hose and breathing mask. The first housing has first and second chambers which are separated by a barrier comprising a carbon dioxide scrubber. The breathing apparatus is configured to conduct hypoxic therapy and tailor the therapeutic session to the patient while collecting data about patient response.

### CROSS-REFERENCE TO RELATED APPLICATIONS

Not Applicable.

### STATEMENT REGARDING FEDERALLY SPONSORED RESEARCH

Not Applicable.

### BACKGROUND

Research has shown that modified air mixtures, different from normal dry air, can be utilized to provide therapeutic outcomes upon inhalation. For example, exposing a patient to oxygen-reduced mixtures can elicit an increase in red blood cells.

Known apparatus and methods for producing hypoxic air suffer from a variety of deficiencies, however. For example, methods and apparatus relying on pressure swing adsorption tend to be expensive; noisy, due to the presence of an air compressor; and heavy. Further, they may not be particularly good at altering carbon dioxide levels. Methods and apparatus relying on hollow fiber membrane filtration tend to provide uncomfortably low humidity and require a humidifier; they also tend to be loud due to the presence of an air compressor.

Methods and apparatus relying simply on reducing the percentage of oxygen in an air mixture by increasing the percentage of another gas, for example nitrogen (referred to as “nitrogen dilution” or “nitrogen enrichment”), to create a hypoxic mixture also suffer from certain deficiencies. In particular, it can be expensive to repeatedly fill a compressed nitrogen tank. Further, the nitrogen tanks can be bulky and heavy.

Hypobaric chambers, in turn, also tend to be large, expensive, and heavy. Further, they are more likely to cause side-effects associated with altitude sickness and cannot easily be cycled quickly between hypoxic and normoxic mixtures.

Finally, existing methods and apparatus relying on rebreathing, while presenting some benefits over other methods and apparatus for creating hypoxic air mixtures, also suffer from certain undesirable characteristics. In particular, a patient's exhalent tends to contaminate the inner workings of such apparatus; additionally, humidity buildup in the respiratory circuit can result in unsanitary conditions.

In light of the foregoing, there remains a need for economical apparatus and method to provide a hypoxic air mixture for the purposes of therapeutic treatment.

All US patents and applications and all other published documents mentioned anywhere in this application are incorporated herein by reference in their entirety. In particular, the following US patents are incorporated by reference:

U.S. Pat. Nos. 5,988,161; 6,561,185; 6,820,619; and 6,997,180, having inventor Mark W. Kroll.

Without limiting the scope of the invention a brief summary of some of the claimed embodiments of the invention is set forth below. Additional details of the summarized embodiments of the invention and/or additional embodiments of the invention may be found in the Detailed Description of the Invention below.

A brief abstract of the technical disclosure in the specification is provided as well only for the purposes of complying with 37 C.F.R. §1.72. The abstract is not intended to be used for interpreting the scope of the claims.

### BRIEF SUMMARY

In some embodiments, a breathing apparatus comprises a breathing mask and a hose attached to the breathing mask. The hose defines an expiratory flow passage through which expiratory gas from the breathing mask flows. The hose is attached to a first port, which defines an inlet into a first housing, such that the flow passage is in fluid communication with the first port. The first port comprises a first directional valve for one-directional flow of the expiratory gas through the first port and into the first housing. The first housing has a first chamber and a second chamber. The second chamber is separated from the first chamber by a barrier. The barrier comprises a carbon dioxide scrubber through which the expiratory gas flows after entering the first chamber. The breathing apparatus further comprises an expandable reservoir in fluid communication with the first chamber, second chamber, or both. The barrier separating the first and second chambers has an ambient air valve and a second port. The second port comprises a second directional valve for one-directional flow of gas out of the second chamber. The ambient air valve is configured to selectively permit entry of ambient air into the second chamber. The second port communicates with the breathing mask via an inspiratory flow passage.

In some embodiments, a method of providing hypoxic therapy to a patient comprises providing a breathing apparatus having a breathing mask, a cartridge assembly, a control assembly, and a hose attached to the breathing mask and cartridge assembly. The method further comprises providing a gaseous mixture to the patient with the breathing apparatus and monitoring the gaseous mixture with the breathing apparatus. The method further includes measuring the patient's oxygen saturation level, measuring the rate of change of the patient's oxygen saturation level, and adjusting the contents of the gaseous mixture provided to the patient with the breathing apparatus.

### BRIEF DESCRIPTION OF THE DRAWING(S)

FIG. 1 shows a graphical representation of a hypoxic therapy treatment session.

FIG. 2 shows graphical representation of another hypoxic therapy treatment session.

FIG. 3 shows graphical representation of another hypoxic therapy treatment session.

FIG. 4 shows a graphical representation of linear and non-linear responses, respectively.

FIG. 5 shows a graphical representation of the oxygen modulation as it nears the target oxygen desaturation.

FIG. 6A/6B shows an embodiment of a logic circuit.

FIG. 7 shows a graphical representation of a single hypoxic period.

FIG. 8 shows a perspective view of an embodiment of a breathing apparatus 10.

FIG. 9 shows another perspective view of the embodiment of FIG. 8.

FIG. 10 shows a partial sectional view of the embodiment of FIG. 8.

FIG. 11 shows a partial sectional view of the embodiment of FIG. 8.

FIG. 12 shows a partial sectional view of the embodiment of FIG. 8.

FIG. 13 shows a schematic view of an embodiment of a breathing apparatus 10.

### DETAILED DESCRIPTION

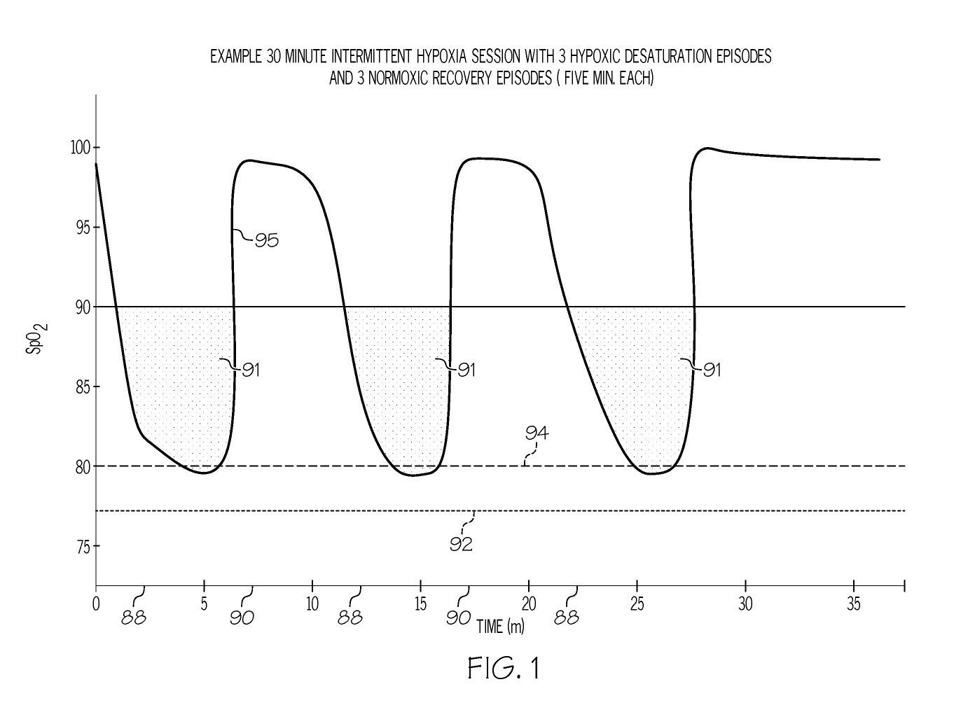

In some embodiments, a therapeutic treatment is administered to a patient via intermittent hypoxic periods. FIG. 1 illustrates an example of a hypoxic therapy treatment session lasting approximately 30 minutes and having three periods of hypoxic desaturation 88 interspersed with periods of normoxic recovery 90. As shown in FIG. 1, each of the periods is approximately five minutes in duration and the patient's arterial oxygen saturation (SpO₂) is labeled 95. During the periods of normoxic recovery 90 the arterial and tissue oxygen concentrations rise. Further, the periods of hypoxia 88 trigger a desired physiological adaptation resulting in a therapeutic response, while the periods of normoxic recovery 90 tend to mitigate side effects of the hypoxia and may further provide a synergistic complement to the periods of hypoxia.

In some embodiments, the treatment regime will include a plurality of sessions (for example 10 or more), each having a duration of between approximately 20 minutes and 8 hours. Within each of the sessions, the hypoxic periods 88 will last approximately 1-8 minutes and be interspersed with periods of recovery 90 lasting from 3 minutes to 16 hours, for example. Of course, other regimens are also suitable, depending upon the patient, condition, and physiologic response.

The vertical axis of the graph of FIG. 1 shows the percentage (%) of arterial oxygen saturation (SpO₂). Generally, hypoxia takes place only when the arterial oxygen saturation of a patient drops below 90%; nonetheless, the threshold value is expected to be patient dependent and the treatment regime can be configurable to have a variable threshold. As shown, the target desaturation is 80%, as illustrated via the horizontal line 94.

During the therapeutic treatment session, the air composition of the air inspired by a patient, along with certain physiological indices, such as heart rate and oxyhemoglobin saturation, may be monitored. In order to illicit the desired therapeutic response from a patient, and to prevent injury, the patient may also be fitted with one or more pieces of monitoring equipment, including but not limited to, a spirometer, pulse oximeter, and sensor(s) to determine exhaled metabolites (e.g., nitric oxide, ventilation rate, expiratory air pressure, inspiratory air pressure, oxygen content, and carbon dioxide content). Further, in some embodiments, the patient and/or supervisor will be given a manual abort switch to end the therapeutic session or a portion of the therapeutic session.

The therapeutic dosage delivered to the patient during each session will be monitored so that the patient receives the prescribed treatment. The dosage of the therapeutic session is defined as the cumulative measure of the Hypoxic Training Index (HTI) as described on pages 589-601 of Intermittent Hypoxia: From Molecular Mechanisms to Clinical Applications by Oleg Bassovitch and Tatiana V. Serebrovskaya (2009), which is herein incorporated by reference. In particular, the HTI is calculated as:

$$HTi = \frac{1}{60} {\int }_{0}^{1} [ 90 - Sp {O}_{2} ( t ) ] d t$$

where HTI is the hypoxic training index, t is the period of time, in seconds, and SpO₂ is the percentage (%) of arterial oxygen saturation, measured at one-second intervals.

As further shown in FIG. 1, the hypoxic dosage 91 begins when the level of oxygen saturation (SpO₂) drops below 90% and ends when the level of oxygen saturation (SpO₂) again reaches 90%. The minimum acceptable level 92 of oxygen saturation (SpO₂) may be set, for example, at 78%. The hypoxic dosage of the therapeutic session is the sum of each of the hypoxic dosages 91.

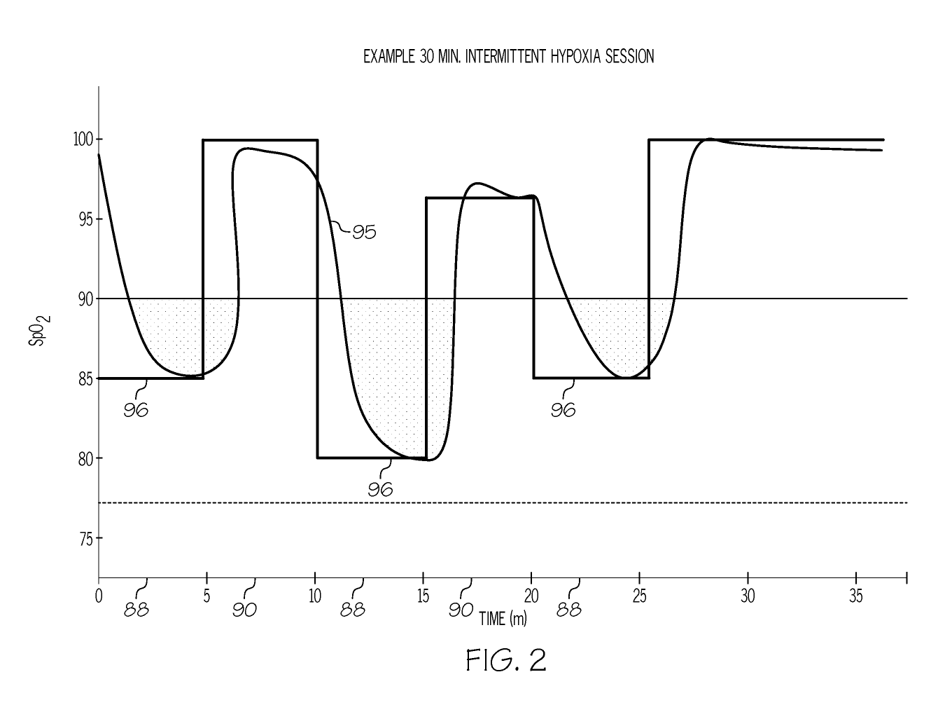

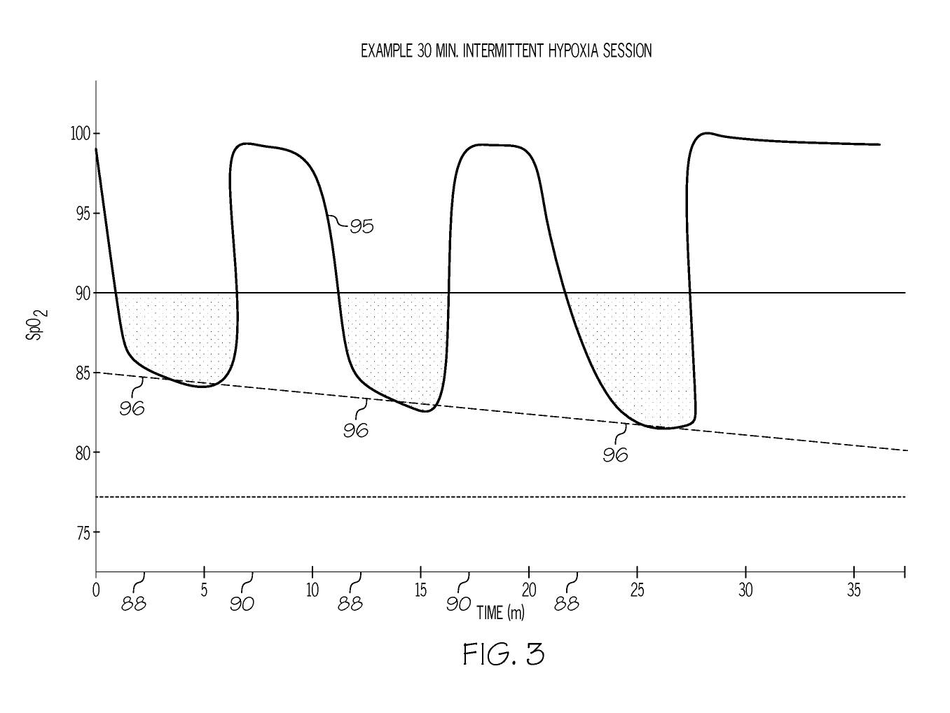

Turning to FIG. 2, it may be desirable to administer varying oxygen desaturation targets 96 during a single session. For example, the hypoxic periods 88 have desaturation targets varying between 80% and 85%, though any desired oxygen saturation may be selected for each hypoxic period 88. In FIG. 3, for example, each successive hypoxic period 88 of a single session has a reduced desaturation target 96 (e.g., 84%, 83%, 82%), which, as shown, follows a line having a negative slope.

In some embodiments, a controller (e.g., programmable logic controller, computer) is used to administer the therapeutic treatment and react as the patient's oxygen saturation nears the desaturation target 96. For example, the controller (e.g., breathing apparatus 10, discussed below) may employ an algorithm able to administer smaller changes in the mixture of gases as the patient nears the desaturation target 96. Additionally, in some embodiments, the algorithm may analyze the velocity at which the patient's oxygen saturation nears the desaturation target 96. As such, the first derivative of the patient's oxygen saturation (vs. time) is determined so that the algorithm can preemptively adjust the oxygen saturation more quickly and drastically if the patient is more rapidly approaching the desaturation target 96. The algorithm may be further configured to respond more aggressively the further away the patient's oxygen saturation is from the desaturation target 96. For example, if the patient is relatively far from the desaturation target 96, one or more valves or injectors is modulated to reach the desaturation target 96 more quickly; and, as the patient gets closer to the desaturation target 96, one or more valves or injectors reacts more modestly. Finally, in some embodiments, the algorithm is configured to modulate the response of the device administering the therapeutic session for each individual patient, for example, by learning how the patient reacts to changes made to the mixture of gases delivered to the patient.

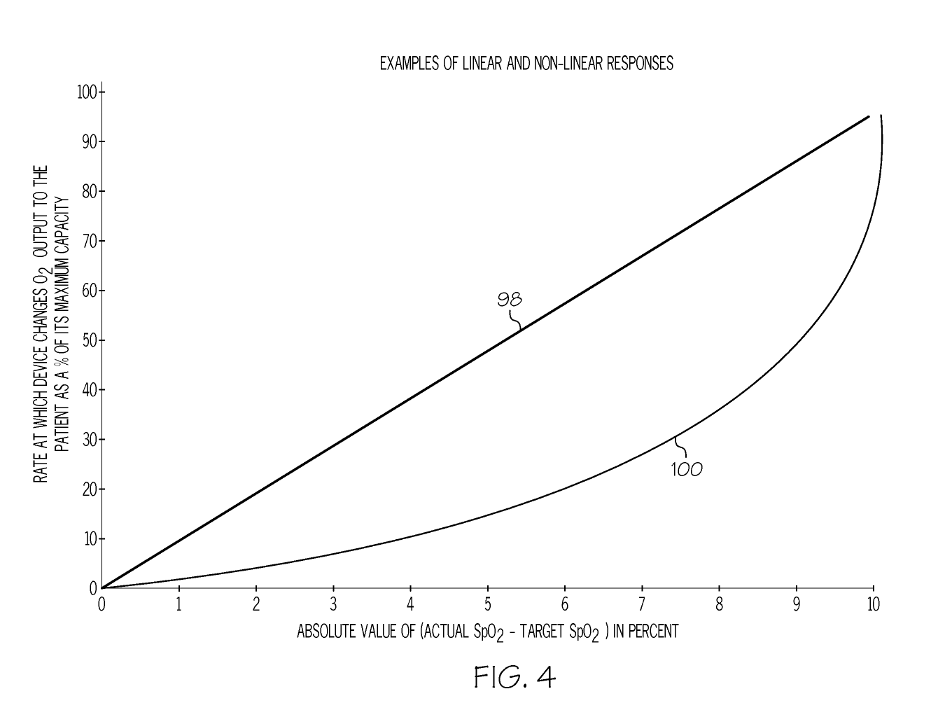

By way of example, one or more valves may be opened or closed to administer the hypoxic therapy. With regard to FIG. 4, a linear response rate of a valve is shown at 98. At 100, a valve response skewed to react more aggressively to oxygen saturation levels that are further from the desaturation target 96 is shown. The horizontal axis shows the absolute value of the percentage difference between the actual oxygen saturation and the target level. The vertical axis shows the rate at which the breathing apparatus 10 changes oxygen output to the patient as a percentage of its maximum capacity. Consequently, as shown via the skewed response curve 100, the larger the difference between the actual saturation and the desaturation target 96, the greater degree the one or more valves will modulate (and/or injectors, etc.) in order to reduce the patient's oxygen saturation. As will be appreciated, the algorithm can further adjust to the patient's individual characteristics and/or session history.

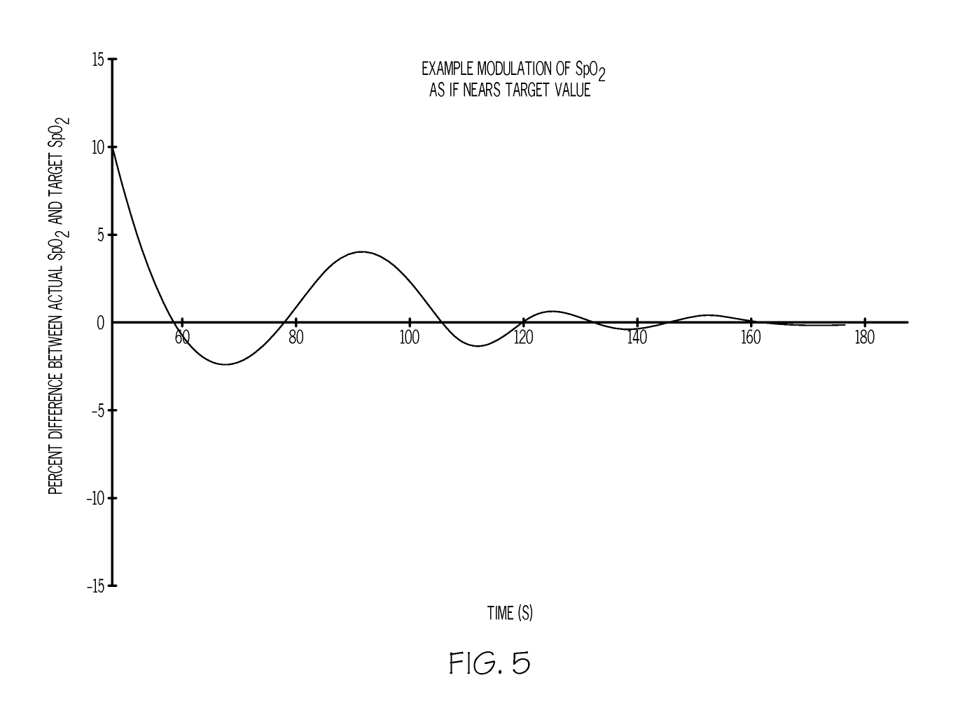

FIG. 5, in turn, shows an example of how a response can be modulated as the oxygen saturation nears the desaturation target 96 (e.g., FIG. 2). The graph shows the percentage difference between the actual oxygen saturation and the desaturation target 96 on the vertical axis and time, in seconds, on the horizontal axis. As will be appreciated, the response may improve with each session and/or hypoxic period, for example, as the algorithm “learns” how the patient reacts to the treatment.

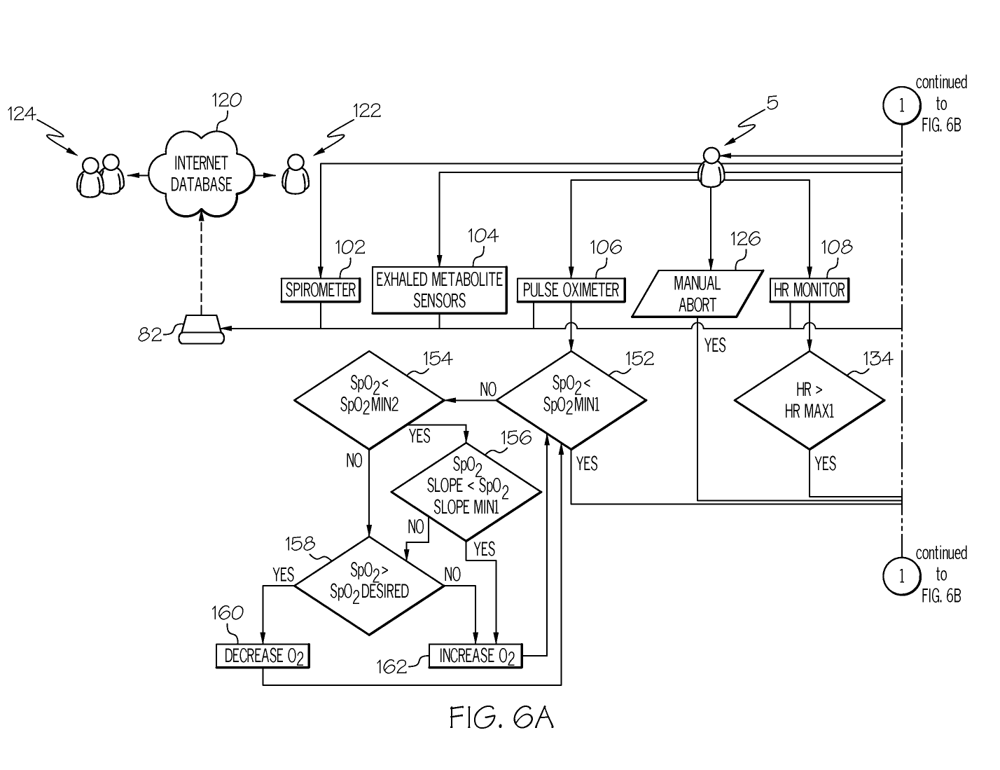

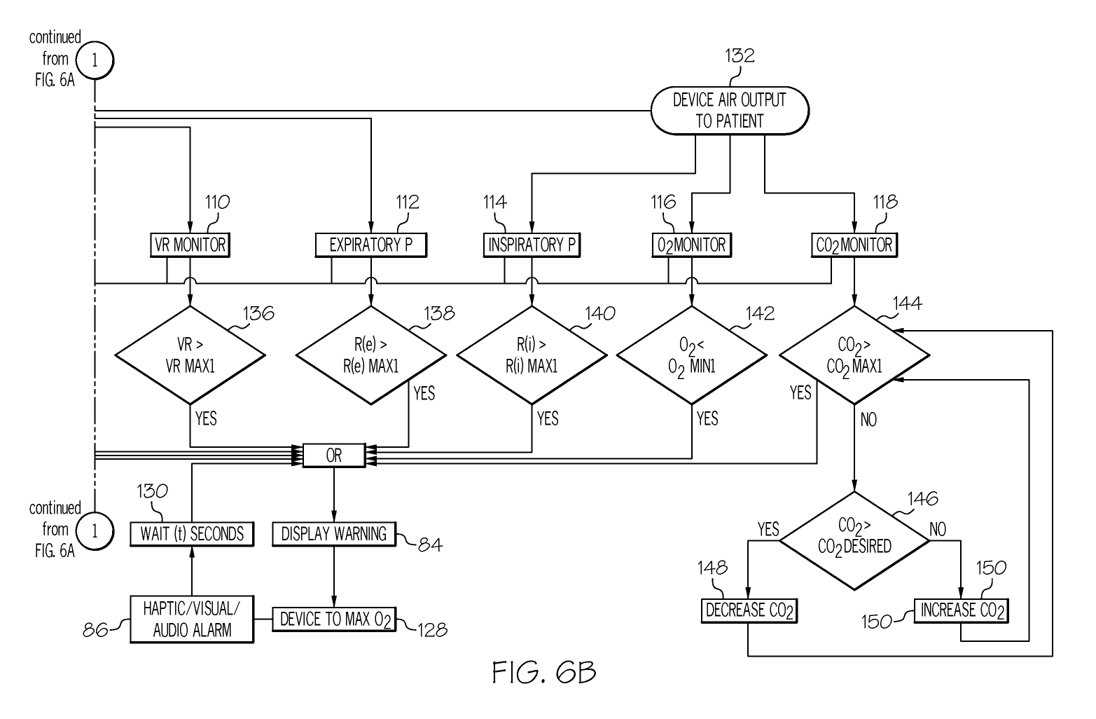

FIG. 6A/6B shows an example of a logic diagram which can be employed to administer the therapeutic hypoxic treatment. The patient 5 may be connected to monitoring equipment which can trigger changes in the treatment administered to the patient via output 132, provide data to a data storage device 82, and/or activate a warning 84 or alarm 86. In particular, as shown in FIG. 6A/6B, the patient 5 is monitored by a spirometer 102, metabolite sensor(s) 104 (monitoring exhaled metabolites), pulse oximeter 106, heart rate monitor 108, ventilation rate monitor 110, and expiratory air pressure monitor 112. In some embodiments, the spirometer 102 can be used to evaluate vital capacity, tidal volume, breathing rate, ventilation rate, and oxygen uptake. Further, in some embodiments, the patient 5 may be prompted during the therapy session, to take a spirometer test. The gaseous output 132 from the device used to administer the therapy (e.g., breathing apparatus 10 as in FIG. 6A/6B) to the patient is monitored via an inspiratory pressure monitor 114, an oxygen monitor 116, and a carbon dioxide monitor 118.

As modified air is delivered to the patient 5, via the output, it is monitored and the data is recorded via the data storage device 82. In some embodiments, and as shown in FIG. 6A/6B, the data storage device 82 records data from each of the spirometer 102, metabolite sensor(s) 104, pulse oximeter 106, heart rate monitor 108, ventilation rate monitor 110, expiratory air pressure monitor 112, inspiratory pressure monitor 114, oxygen monitor 116, and carbon dioxide monitor 118. Further, in some embodiments, the data is transmitted to an internet database 120 where it can be reviewed by the prescribing physician 122 and/or the manufacturer 124.

In some embodiments, a tissue oxygen saturation monitor and a noninvasive cardiac function monitor are also included to monitor the patient and provide feedback.

In some embodiments, the patient 5 is provided with a manual abort function 126 which, upon activation, will end the therapeutic session or a period of the therapeutic session.

With further reference to FIG. 6A/6B, the level of oxygen delivered to the patient 5, for example via output 132, is based on a number of parameters. Additionally, algorithm and device are vigilant to maintain safety minimums at all times during the therapeutic session. For example, if the patient 5 triggers the manual abort function 126, aborting the session, a warning 84 will be displayed and the device will be set to provide maximum oxygen 128; further, a haptic, visual, and/or audio alarm 86 will be employed; and the algorithm will wait for a period of time 130 (e.g., 120 seconds) before continuing the therapeutic session, provided all measures are within acceptable levels.

If the heart rate monitor 108 detects that the patient 5 has a heart rate greater than (or equal to) a predetermined threshold (e.g., HR MAX1 in FIG. 6A), shown at 134, which may be entered at the beginning of the session, the device (e.g., breathing apparatus 10) will display a warning 84; provide maximum oxygen 128; provide a haptic, visual, and/or audio alarm 86; and wait for a period of time 130 before continuing the session, provided all measures are within acceptable levels, as discussed above with the respect to the manual abort function 126.

The max heart rate threshold will depend upon the excursion level of the patient 5. In some embodiments, the max heart rate threshold for treatments conduct while the patient 5 is at rest will be 110-120 beats per minute. In some embodiments, where the hypoxia treatment is combined with exercise, the max heart rate (MHR) will be equal to 220 minus the age of the patient:

MHR=220−patient age

Further, if the ventilation rate monitor 110 detects that the patient 5 has a ventilation rate above (or equal to) a predetermined threshold (e.g., VR MAX1 in FIG. 6B), shown at 136, which may be entered at the beginning of the sessions, the device will display a warning 84; provide maximum oxygen 128; provide a haptic, visual, and/or audio alarm 86; and wait for a period of time 130 before continuing the session, provided all measures are within acceptable levels, as discussed above with the respect to the manual abort function 126.

In a similar fashion, if the expiratory air pressure monitor 112 detects that the expiratory pressure from the patient 5 is greater than (or equal to) a maximum threshold (e.g., R(e) MAX1), shown via 138, the device will display a warning 84; provide maximum oxygen 128; provide a haptic, visual, and/or audio alarm 86; and wait for a period of time 130 before continuing the session, provided all measures are within acceptable levels, as discussed above with the respect to the manual abort function 126. In some embodiments, the device (e.g., breathing apparatus 10) is able to regulate and increase the expiratory pressure; in some embodiments, the maximum threshold is determined by the algorithm. In some embodiments, in situations where the algorithm calls for “normal” expiratory resistance, for example, the maximum threshold will be between zero (0) and ten (10) mm of H₂O.

If the inspiratory air pressure monitor 114 detects that the inspiratory air pressure is greater than (or equal to) a maximum threshold (e.g., R(i) MAX1), shown via 140, the device will display a warning 84; provide maximum oxygen 128; provide a haptic, visual, and/or audio alarm 86; and wait for a period of time 130 before continuing the session, provided all measures are within acceptable levels, as discussed above with the respect to the manual abort function 126. In some embodiments, the device is able to regulate and increase the inspiratory pressure; in some embodiments, the maximum threshold is determined by the algorithm. In some embodiments, in situations where the algorithm calls for “normal” inspiratory pressure, for example, the maximum threshold will be between zero (0) and ten (5) mm of H₂O.

If the oxygen monitor 116 detects that the oxygen partial pressure of the air delivered to the patient is less than (or equal to) a minimum threshold (e.g., O₂ MIN1), shown at 142, a warning 84 is displayed; maximum oxygen 128 is set; a haptic, visual, and/or audio alarm 86 response is provided; and the algorithm waits a period of time 130 before continuing the session, provided all measures are within acceptable levels, as discussed above with the respect to the manual abort function 126. In some embodiments, the prescribing physician 122 and/or manufacturer 124 is able to change the oxygen partial pressure minimum threshold. In some embodiments, the oxygen partial pressure minimum threshold is approximately 70 mm Hg-100 mm Hg or the partial pressure corresponding to 9%-13% oxygen by volume.

If the carbon dioxide monitor 118 detects that the carbon dioxide partial pressure of the air delivered to the patient is greater than (or equal to) a maximum threshold (e.g., CO₂ MAX1), shown at 144, a warning 84 is displayed; maximum oxygen 128 is set; a haptic, visual, and/or audio alarm 86 is elicited; and the algorithm waits for a period of time 130 before continuing the session, provided all measures are within acceptable levels, as discussed above with the respect to the manual abort function 126. In some embodiments, the levels of carbon dioxide administered for inspiration may be altered; in some embodiments, under no situation will the level of carbon dioxide exceed 3% by volume at 1 atmosphere, corresponding to 22.8 mm Hg of carbon dioxide.

If the carbon dioxide monitor 118 detects that the carbon dioxide partial pressure of the air delivered to the patient is less than the maximum threshold, shown at 144, the algorithm will then determine whether the carbon dioxide partial pressure of the air delivered to the patient is greater than (or equal to) the desired level, shown at 146. If the carbon dioxide level is greater than (or equal to) the desired level, the algorithm 10 will decrease the carbon dioxide level, shown at 148. Alternatively, if the carbon dioxide level is less than the desired level, the algorithm 10 will increase the carbon dioxide level, as shown at 150. After determining whether to increase the carbon dioxide level 150 or decrease the carbon dioxide level 148, the logic circuit loops back to 144 to again determine whether level of carbon dioxide is in an appropriate range.

With reference to FIGS. 6A/6B and 7, the pulse oximeter 106 measures the level of oxygen saturation in the patient's blood (referred to as “SpO₂”). If the level of oxygen saturation drops below (or is equal to) a predetermined threshold (e.g., SpO₂ MIN1), for example, the minimum acceptable level 92 (e.g., 78%, 79%, 80%), as shown at 152, a warning 84 is displayed; maximum oxygen 128 is set; a haptic, visual, and/or audio alarm 86 is provided; and the algorithm waits for a period of time 130 before continuing the session, provided all measures are within acceptable levels, as discussed above with the respect to the manual abort function 126. In some embodiments, the prescribing physician 122 and/or manufacturer 124 is able to set the minimum acceptable level 92. In some embodiments, the minimum acceptable level 92 is set between 70% and 85% oxygen saturation, for example in 1% increments (70%, 71%, 72% . . . 85%).

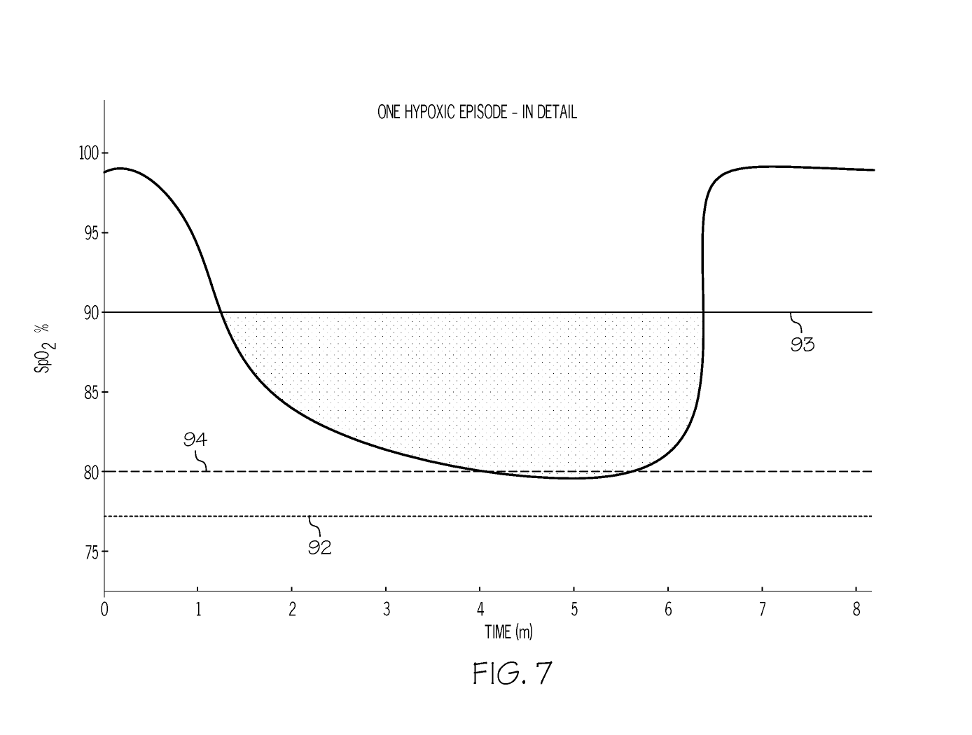

If, however, the patient's oxygen saturation is greater than the minimum acceptable level 92, the algorithm determines whether the patient's oxygen saturation is less than (or equal to) a set value 93 (FIG. 7) (e.g., SpO₂ MIN2), as shown at 154 (FIG. 6A/6B). In some embodiments, the set value 93 is equal to the “hypoxic threshold” (e.g., 85%, 86%, 87%, 88%, 89%, 90%). Once the patient's oxygen saturation crosses below the set value 93, the algorithm is more vigilant to guard against rapid oxygen desaturation even though the patient's oxygen saturation level may be above the target desaturation 94. If the patient's oxygen saturation is less than or equal to the set value 93, then the algorithm determines whether the rate of change (slope of oxygen saturation vs. time) of the patient's oxygen saturation is less than or equal to a predetermined value (e.g., SpO₂ SLOPE MIN1), as shown at 156. In some embodiments, the rate of change of the patient's oxygen saturation level should not exceed negative ten (−10) over a two (2) second interval.

If the rate of change of the patient's oxygen saturation is less than or equal to the predetermined value, the relative oxygen supply to the patient 5 is increased, as shown at 162. Alternatively, if the rate of change of the patient's oxygen saturation is greater than the predetermined value, as shown at 156, the algorithm then determines whether the patient's oxygen saturation level is greater than or equal to the desired oxygen saturation, as shown at 158 in FIG. 6A/6B, which may vary during the therapy session (e.g., depending upon whether the patient is undergoing a hypoxic period 88 or normoxic period 90).

Moreover, and returning to reference 154, if the patient's oxygen saturation is determined to be greater than the set value 93, the algorithm then determines if the patient's oxygen saturation is greater than or equal to the desired oxygen saturation, as shown at 158 in FIG. 6A/6B, which may vary during the therapy session (e.g., depending upon whether the patient is undergoing a hypoxic period 88 or normoxic period 90). If the patient's oxygen saturation is greater than or equal to the desired oxygen saturation, the relative oxygen supply to the patient is decreased, as shown at 160.

Whether the oxygen supply to the patient 5 is increased via 160 or decreased via 162, the logic circuit loops back to 152 to again determine whether the patient's oxygen saturation is less than or equal a predetermined threshold, for example, the minimum acceptable level 92.

At the conclusion of each therapy session, in some embodiments, the recorded data is uploaded to the internet database 120 where the manufacturer 124 and/or prescribing physician 122 can evaluate patient compliance, response, and device settings for any desired modifications. In some embodiments, the device will provide the physician and, in some embodiments, the device manufacturer with response information from the patient so that the information can be analyzed to provide more efficient and better tailored sessions, both by the physician and device manufacturer. In some embodiments, the controller used to control and administer the therapeutic treatment will employ feedback from patient conditions to adjust the therapeutic treatment in real time and predict the patient's response (e.g., physiologic response) to the treatment. For example, in some embodiments, the controller is configured to adjust the treatment by monitoring patient physiology and preempt undesirable physiologic conditions that may be approaching the threshold boundary. Further, the rate at which the physiologic condition is approaching the threshold boundary can be used to illicit a change in the therapeutic treatment; for example, the controller may take into account the rate of change of arterial oxygen saturation with respect to time and adjust the therapy depending upon the rate of change.

In some embodiments, the controller can utilize data obtained from a previous therapeutic session for a particular patient to customize the therapy. Further, the controller can utilize data from the entire patient population (e.g., obtained via a network connection) and cross reference physiological inputs to customize the therapy.

In some embodiments, the controller is programmed to incorporate and administer doses based on one or more dosing conventions (e.g., Hypoxic Training Index) and incorporate and administer one or more diagnostic protocols (e.g., hypoxic challenge protocol). The diagnostic data can further be input as a variable in the control algorithm. In some embodiments, a physician or user can administer a predetermined dosage of hypoxic therapy by simply entering the desired dosage into the controller (e.g., control assembly 12, discussed below).

In some embodiments, the controller includes one or more proportional-integral-derivative (PID) controllers to control various aspects of the therapy. For example, the controller can control one or more valves (as described in greater detail below, for example) based on the patient's physiological conditions, for example, arterial oxygen saturation. In some embodiments, the PID is configured as follows:

$${C}_{v} = {w}_{1} Δ + {w}_{2} \frac{d Δ}{d t} + {w}_{3} \int Δ d t$$

where Cᵥ sets the position of a control valve, w₁, w₂, and w₃ are weighting factors, and τ is the time constant. Further,

Δ=oxygenᵢ−oxygenₜ

or, the difference between the instantaneous oxygen saturation and the target oxygen saturation.

In some embodiments, the time constant, τ, compensates for a lag between the reading taken by the pulse oximeter and the patient's breathing. In some embodiments, τ is 60 seconds, though other values can be used, depending upon the instruments used to record data and the device used to administer the hypoxic therapy.

In some embodiments, the controller is configured to automatically adjust the weighting factors (w₁, w₂, and w₃) based on physiologic feedback from the patient and other data sources discussed above in order to minimize error in the PID controller's response.

The PID can be used in conjunction with a fully closed-loop device for administering therapy or a partially closed-loop device. A partially closed-loop device, for example, can include one or more ambient air ports, as discussed in greater detail below.

Additionally, it will be appreciated that delivery of the hypoxic therapy involves a non-linear system, for which the PID controller is appropriately configured.
The system and method described above can be used in conjunction with any suitable device for administering therapy, for example, pressure swing adsorption devices, vacuum chamber devices, nitrogen enrichment devices, hallow fiber membrane devices, and rebreathing devices.

The following discussion is directed to embodiments of suitable devices for administering hypoxic therapy. In at least some embodiments, these devices are rebreathing devices. In some embodiments, a breathing apparatus 10 comprises a control assembly 12 and a cartridge assembly 14. In some embodiments, the cartridge assembly 14 is disposable and is attached to the control assembly 12 in an easily removable fashion, for example via clips, retainers, or any suitable fastener.

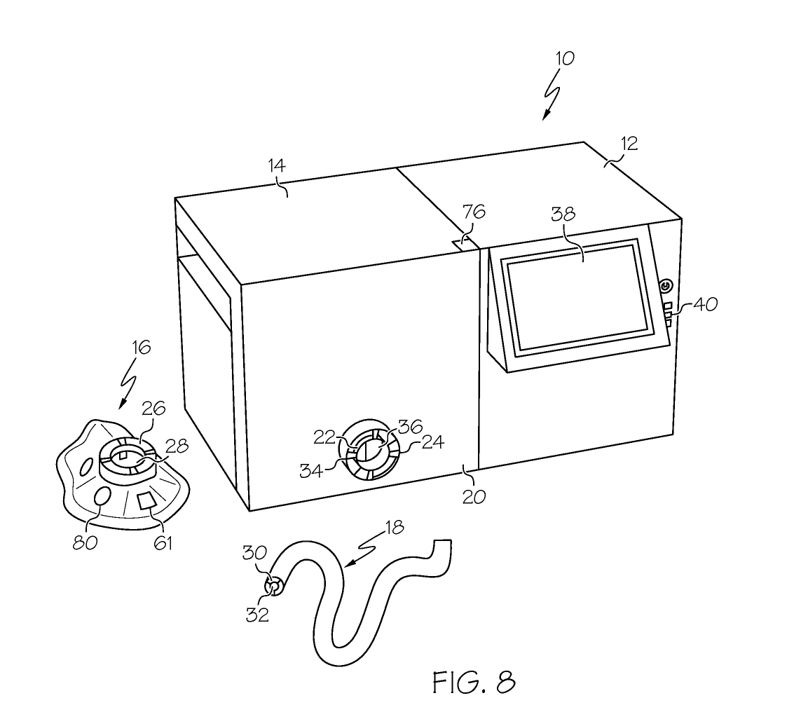

In some embodiments, the cartridge assembly 14 comprises a first housing 20. The first housing 20 has a first port 22 and a second port 24. In some embodiments, for example as shown in FIG. 8, the first and second ports 22, 24 are coaxial.

The breathing apparatus 10 further comprises a breathing mask 16 and a hose 18 which can be attached to the mask 16. In some embodiments, the breathing mask 16 comprises an expiratory flow passage 26 and an inspiratory flow passage 28. As shown in FIG. 8, for example, in some embodiments, the expiratory flow passage 26 and inspiratory flow passage 28 are coaxial. As further shown in FIG. 8, the expiratory flow passage 26 is outside of the inspiratory flow passage 28. The expiratory flow passage 26 can also be configured to be inside of the inspiratory flow passage 28.

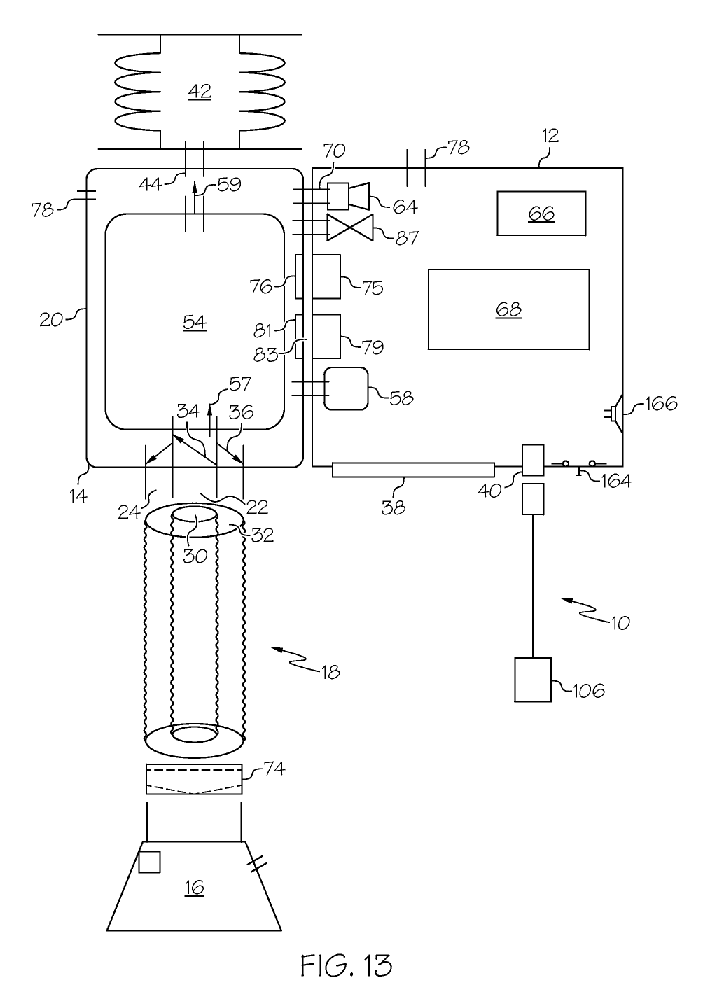

The hose 18 further comprises an expiratory flow passage 30 and an inspiratory flow passage 32 which, in some embodiments, are coaxial. In some embodiments, the expiratory flow passage 30 is outside of the inspiratory flow passage 32; in some embodiments, for example as shown in FIG. 13, the expiratory flow passage 30 is inside the inspiratory flow passage 32.

The hose 18 serves to connect the mask 16 with the first housing 20 such that expiratory gas from a user flows through the expiratory flow passages 26 of the mask 16, the expiratory flow passage 30 of the hose 18, through the first port 22, and into the cartridge assembly 14. As will further be appreciated, the hose 18 further connects the inspiratory flow passages 28, 32 with the second port 24.

In some embodiments, the first port 22 comprises a first directional valve 34, which permits expiratory gas to flow into the cartridge assembly 14 through the first port 22 but prevents flow in the opposite direction. Further, in some embodiments, the second port 24 includes a second directional valve 36, which permits inspiratory gas to flow out of the cartridge assembly 14 through the second port 24 but prevents flow in the opposite direction.

The control assembly 12 comprises, in some embodiments, a display 38 and at least one data port 40. In some embodiments, the display 38 has a touchscreen. The touchscreen can be used to configure the breathing apparatus 10 and change settings, as discussed below in greater detail. The touchscreen can take on any desirable configuration, for example, it can be a resistive touchscreen, capacitive touchscreen, infrared touchscreen, or surface acoustic wave screen. Further, the data port(s) 40 can be used to program and/or download data from the breathing apparatus 10.

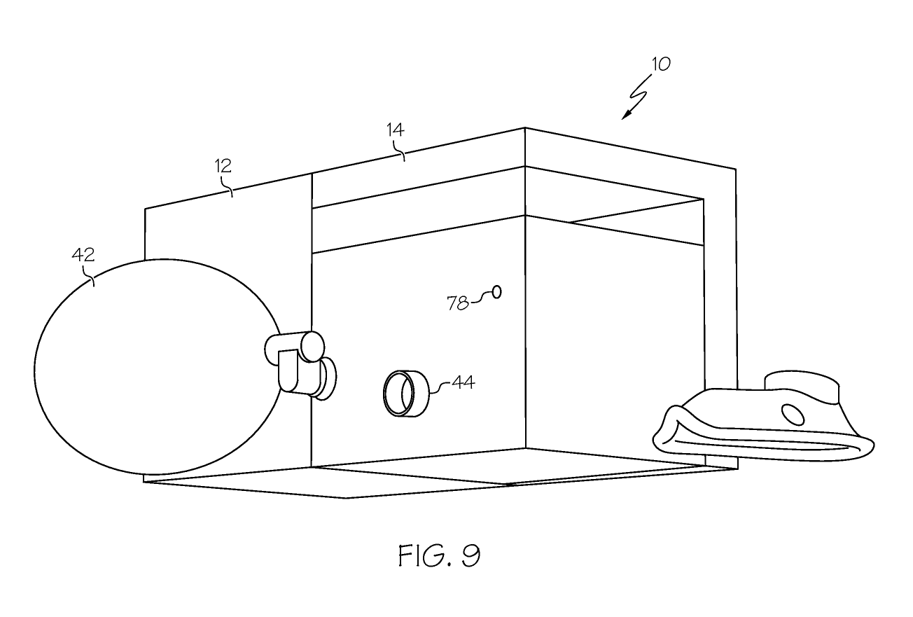

With regard to FIG. 9, in some embodiments, the breathing apparatus 10 further comprises an expandable reservoir 42 (shown detached from the cartridge assembly 14). In operation, the expandable reservoir 42 is attached to a third port 44 of the cartridge assembly 14. The expandable reservoir 42 acts as a counter-lung as the user breathes.

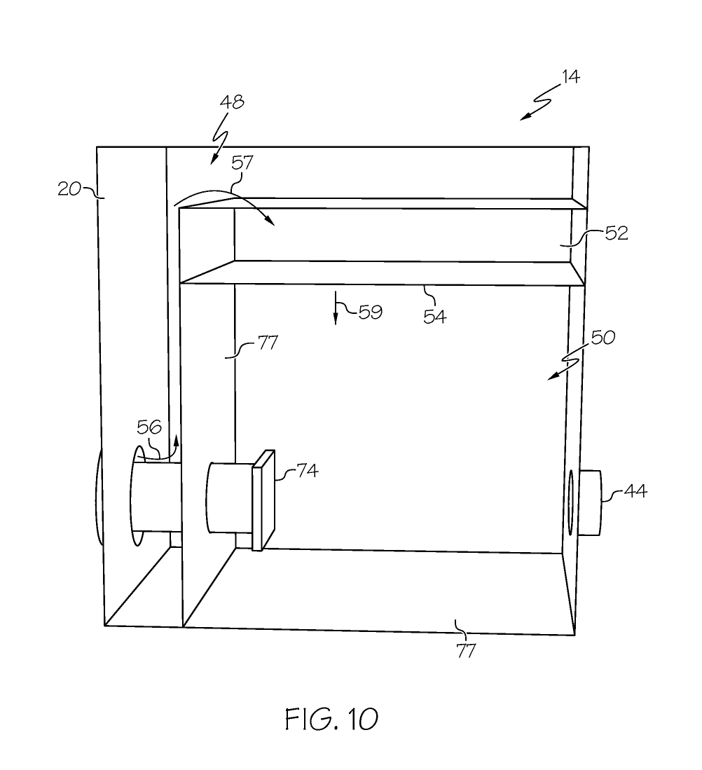

Referring to FIG. 10, the cartridge assembly 14 is shown in a cutaway view. The cartridge assembly 14 has a first housing 20 defining a first chamber 48. In some embodiments, the first housing 20 further defines a second chamber 50 which, in some embodiments, is located at least partially within the first chamber 48. The first and second chambers 48, 50 are separated by a barrier 52. In some embodiments, the barrier 52, or at least a portion thereof, comprises a carbon dioxide scrubber 54 which removes carbon dioxide from the gas flowing from the first chamber 48 to the second chamber 50. The carbon dioxide scrubber 54 can comprise any suitable material and, in some embodiments, it includes lithium chloride, lithium hydroxide, calcium hydroxide, and or soda lime. In some embodiments, at least a portion of the barrier 52 is at least semi-permeable (e.g., the carbon dioxide scrubber 54), while at least a portion of the barrier 52 is non-permeable to the extent that gases cannot freely flow through such portions of the barrier 52 (e.g., the non-permeable portions comprise a wall). In some embodiments, the carbon dioxide scrubber 54 further includes an indicator, for example an ethyl violet indicator, to show when it has become saturated and is in need of replacement, recharging, or cleaning.

In some embodiments, expiratory gas flows out of the breathing mask 16 into the hose 18 and, subsequently, through the first port 22 (FIG. 8) into the first chamber 48, as illustrated via arrow 56. Upon entering the first chamber 48, the expiratory gas flows through the carbon dioxide scrubber 54 and enters the second chamber 50, as shown via flow arrows 57 and 59. Inside the second chamber 50, also referred to as the mixing chamber, the gas entering the second chamber 50 via the carbon dioxide scrubber 54 mixes with the gas already in the second chamber 50 and some fraction of the mixed gas flows into the expandable reservoir 42 via the third port 44. The amount of gas flowing into the expandable reservoir 42 can be regulated by the extent to which the expandable reservoir 42 is compressed and expanded. It will be appreciated that the expandable reservoir 42 does not have to be attached to the second chamber 50. Instead, it can also be in fluid communication with the first chamber 48, in which case, the gas mixture in the expandable reservoir 42 will not be scrubbed by the carbon dioxide scrubber 54. The expandable reservoir 42 can be in fluid communication with the first chamber 48, second chamber 50, or both the first and second chambers 48, 50.

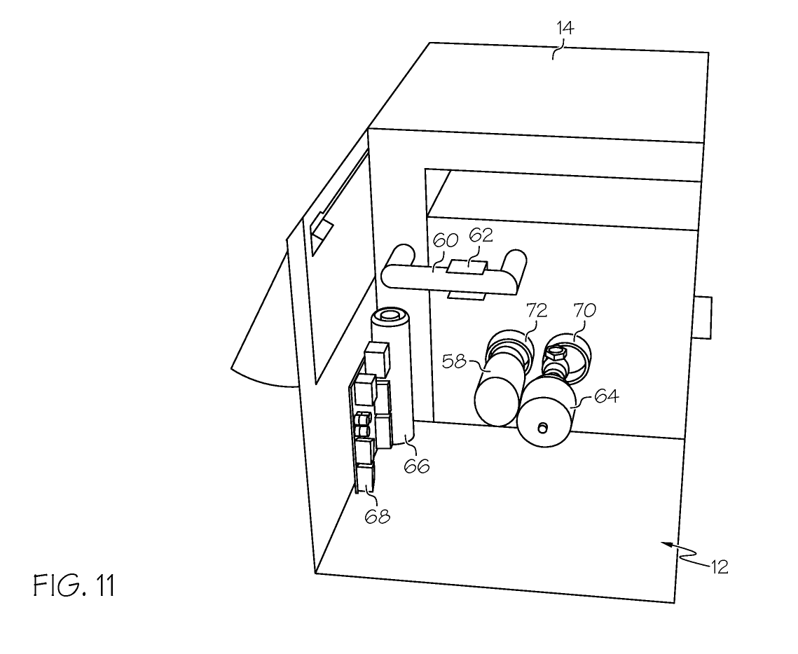

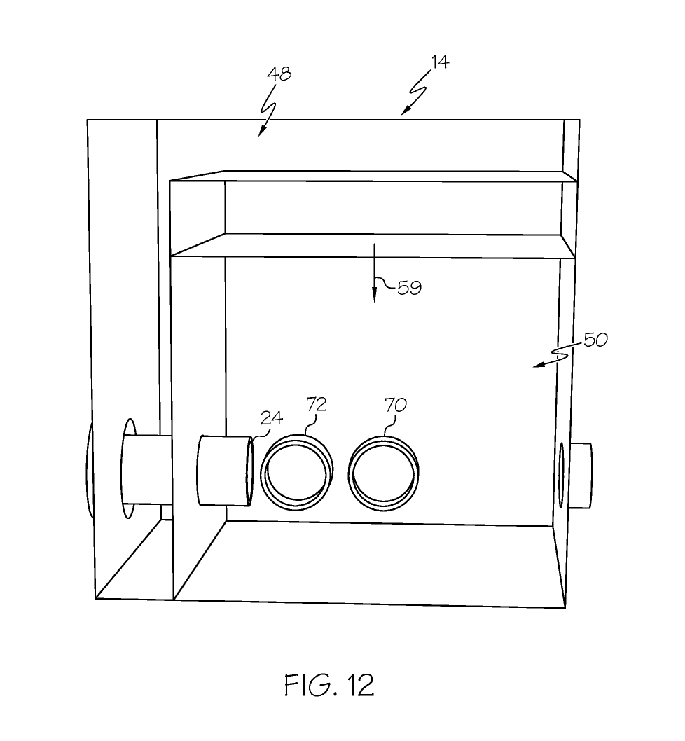

In some embodiments, the second chamber 50 has one or more sensors to measure the components of the gas within the chamber. In some embodiments, one or more of the sensors can be located on a single mount, probe, or other tool that extends into the second chamber 50. As shown in FIG. 11, for example, a sensor 58 extends at least partially into the second chamber 50 (FIG. 12) from the control assembly 12. In some embodiments, the sensor 58 includes an oxygen sensor to measure the oxygen concentration in the second chamber 50. The sensor 58 can further include a chemo-electric gas sensor and/or one or more solid semi-conductors. In some embodiments, the sensor 58 is an optical gas sensor or analyzer.

In some embodiments, for example where a single sensor unit is used to measure oxygen and carbon dioxide, such as sensor unit employs electrode based analysis or optode based analysis. Where optode based gas measurement is employed, sensor is configured to measure florescent decay. Consequently, where optical sensing is used, the sensor does not need to extend into the second chamber 50. Instead, the sensor merely needs to be able to optically view the gas in the chamber (e.g., second chamber 50), for example through a material that is sufficiently transparent to the sensor.

In some embodiments, the one or more sensors can measure the components of the gas within the first chamber 48 in lieu of or in addition to measuring the components of the gas within the second chamber 50.

As further shown in FIG. 11, in some embodiments, the breathing apparatus 10 comprises a barrier bypass 60. The barrier bypass 60 permits gas to flow from the first chamber 48 into the second chamber 50 (FIG. 10) without passing through the carbon dioxide scrubber 54. In some embodiments, the barrier bypass 60 includes a regulator valve 62 to regulate the amount of carbon dioxide that bypasses the carbon dioxide scrubber 54. In some embodiments, the regulator valve 52 comprises a solenoid valve. In some embodiments, however, the breathing apparatus 10 does not have a barrier bypass 60, however.

In some embodiments, the breathing apparatus 10 further comprises an ambient air injector 64 or ambient air valve, a power storage unit (e.g., rechargeable or non-rechargeable battery) 66, and a circuit board 68. Where a battery is used, it can be of any suitable type, for example lithium ion, nickel cadmium, nickel hydrogen, nickel-metal-hydride. The power storage unit 66 can further comprise a fuel cell, for example a hydrogen fuel cell.

The ambient air injector 64 (e.g., air pump or ambient air valve) is used to add ambient air to the second chamber 50 to achieve the desired gas mixture. In some embodiments, the ambient air injector 64 comprises a valve; in some embodiments, the ambient air injector 64 comprises an air pump which forces ambient air into the second chamber 50. In some embodiments, the ambient air injector 64 injects ambient air through an air injection port 70 in the side of the cartridge assembly 14. In some embodiments, the ambient air injector 64 comprises an air pump that is in fluid communication with the second chamber 50 via a proportional valve having one input and two outputs. In some embodiments, one of the outputs is in fluid communication with the second chamber 50 while the other output is exhausted in such a way as not to be in fluid communication with the respiratory circuit of the breathing apparatus 10. The ambient air injection 64 can further include a combination of an air pump, valve (e.g., solenoid valve), or multiple pumps and/or valves in parallel with one another.

In some embodiments, the ambient air injection 64 is in fluid communication with the first chamber 48 or in fluid communication with both the first and second chambers 48, 50. In some embodiments, the ambient air injection 64 is configured to emit a varying flow of ambient air into the first and/or second chamber 48, 50 in order to create the desired mixture.

Additionally, the sensor 58 at least partially extends into the second chamber 50 (FIG. 12) via sensor port 72. As discussed above, in some embodiments, the sensor 58 need not extend into the second chamber 50, for example where an optical gas analyzer is employed.

The circuit board 68, in turn, controls the various valves and components of the breathing apparatus 10, via a microcontroller, for example, and the power storage unit 66 can provide power to some or all of the systems or it can be used as backup power if primary power to the breathing apparatus 10 is lost or interrupted.

Turning to FIG. 12, the cartridge assembly 14 is shown in a partial cutaway view looking into the first and second chambers 48, 50. Additionally, the air injection port 70 and sensor port 72 are shown; for the purposes of illustration, however, the adjacent wall section is not shown. Inside the second chamber 50, the mixture of gas exiting the carbon dioxide scrubber 54, illustrated via flow arrow 59, mixes with ambient air entering the second chamber 50 through the air injection port 70, if any, gas that is routed through the barrier bypass 60 into the second chamber 50, if any, and gas entering the second chamber 50 through the third port 44 from the expandable reservoir 42 (FIG. 9). Upon mixing, the gas exits the second chamber 50 via the second port 24, which is in fluid communication with the inspiratory flow passage 32 of the hose 18 (FIG. 8). Further, the inspiratory flow passage 32 of the hose 18 is in fluid communication with the inspiratory flow passage 28 of the breathing mask 16.

In some embodiments, the breathing apparatus 10 further comprises a particulate filter 74 (FIG. 10). The particulate filter 74 is located along the inspiratory flow path, for example at the beginning of the inspiratory flow path adjacent to the second port 24, as shown in FIG. 10. In some embodiments, however, the particulate filter 74 is located along a portion of the inspiratory flow passage 32 of the hose 18 or the inspiratory flow passage 28 of the breathing mask 18. The breathing apparatus 10 can also have more than one particulate filter 74. In some embodiments, the particulate filter 74 comprises a HEPA (high efficiency particular air) filter.

In some embodiments, the cartridge assembly 14 comprises a single-use disposable cartridge, including the carbon dioxide scrubber 54, first directional valve 34, second directional valve 36, first and second chambers 48, 50. In this way, there is no need to clean the interior of the first and second chambers 48, 50 or refill the carbon dioxide scrubber 54.

In some embodiments, one or more portions of the cartridge assembly 14 and/or breathing mask 16 and/or hose 18 include anti-microbial linings which, in some embodiments, are located along portions thereof that come into contact with exhaled air from the patient. Anti-microbial linings can also be used on non-disposable portions of the breathing apparatus 10. For example, portions of the control apparatus 12 that come into contact with exhalent can include an anti-microbial lining.

Additionally, in some embodiments, the cartridge assembly 14 includes a RFID (radio frequency identification) chip 76 (FIG. 8) having a unique identifier (e.g., unique serial number). In this way, the cartridge assembly 14 can communicate with the control assembly 12 in order to record the serial number of the cartridge assembly upon use and, in some embodiments, prevent multiple uses of the cartridge assembly 14 or allow for a predetermined number of uses. In this way, the RFID chip provides a safety feature by preventing the use of a cartridge assembly 14 that is depleted, for example. Further, the RFID chip may be used to verify that the cartridge assembly 14 has passed its expiration date and to verify that it is not an imitation product.

In some embodiments, the breathing apparatus 10 includes a near field communicator chip, tag, and/or system. Near field communication can be used to power, read, and write information to an RFID chip, for example. The near field communicator (e.g., RFID tag) can be located as part of the cartridge assembly 14, breathing mask 16, hose 18, and/or any other suitable component.

In some embodiments, the cartridge assembly 14 comprises a plurality of walls 77 which are formed from plastic, sturdy paper product, for example 1-4 mm in thickness, or some combination of plastic and sturdy paper product. In some embodiments, the sturdy paper product is coated with a wax, in order to form a barrier to the gas mixture and any water vapor.

Some embodiments of the breathing apparatus have one or more holes 78 (FIG. 9) configured to allow a small amount of ambient air to enter the inspiratory flow path or second chamber 50 such that in a malfunction scenario, the oxygen concentration of the gas mixture in the inspiratory flow passage 28 of the breathing mask 16 cannot go below a predetermined threshold, for example, 9.5% oxygen by volume of gas mixture. In some embodiments, the one or more holes 78 define an opening in the side of the second chamber 50. In some embodiments, however, the one or more holes 78 defines an opening in the breathing mask 16 or inspiratory flow passage 32 of the hose 18. The one or more holes 78 can also be in the first chamber 48 in lieu of or in addition to the one or more holes 78 in the second chamber 50.

In some embodiments, the breathing apparatus 10 includes a normally open solenoid valve in fluid communication with the second chamber 50 and/or the inspiratory path. In some embodiments, the second directional valve 36 comprises such normally open solenoid valve. In some embodiments, the normally open solenoid valve functions as a safety feature as the valve does not need power to open and, instead, remains open unless it is powered closed.

In some embodiments, any of the valves herein described can include multiple parallel valves for the purpose of redundancy.

In some embodiments, the breathing apparatus 10 includes a reserve carbon dioxide scrubbing cartridge to permit completion of the therapy session in the event the carbon dioxide scrubber 54 (e.g., primary CO₂ scrubber) is exhausted before the therapy session is completed.

In some embodiments, the breathing mask 16 includes an accelerometer 61 or other position sensing device. The accelerometer 61 can be used to verify that the patient is sitting upright. If the accelerometer 61 detects that the patient is in an undesirable position, for example due to fainting, it can restore full oxygen to the patient, for example. In some embodiments, the breathing mask 16 has at least one air intake valve 80 to permit the inflow of ambient air. In some embodiments, the air intake valve 80 comprises a solenoid valve. The air intake valve 80 can take on any suitable structure or configuration; for example, the air intake valve 80 can be a solenoid valve or can be actuated by a shape memory alloy. Further, in some embodiments, it is actuated by a microprocessor that is onboard the breathing mask 16 or otherwise attached to the breathing apparatus 10. In some embodiments, the breathing mask 16 has a safety valve.

In some embodiments, the breathing apparatus 10 includes one or more Universal Serial Bus (USB) ports to communicate with peripheral devices.

With regard to FIG. 13, a schematic diagram of an embodiment of a breathing apparatus 10 is shown having a control assembly 12 and a cartridge assembly 14. The cartridge assembly 14 is attached to a breathing mask 16 via a hose 18 which, as shown, has a coaxial configuration. A particulate filter 74 is disposed along at least one of the inspiratory flow passage 32 and expiratory flow passage 30. The cartridge assembly 14 comprises a first directional valve 34 and a second direction valve 36. In some embodiments, the first directional valve 34 permits flow into the carbon dioxide scrubber 54, as illustrated via flow arrow 57. Gas exits the carbon dioxide scrubber 54, for example into an outer portion (e.g., second chamber 50) of the first housing 20, as illustrated via flow arrow 59. In some embodiments, the breathing apparatus 10 comprises an expandable reservoir coupled to the cartridge assembly 14 via a third port 44. Further, in some embodiments, the cartridge assembly 14 comprises a hole 78, which can act as a safety feature, for example, by preventing the pressure within the first housing 20 from exceeding a predetermined maximum value, or by permitting the introduction of ambient air into the first housing 20.

In some embodiments, the control assembly 12 comprises a display 38 and one or more data ports 40, for example a USB (universal serial bus) port. In some embodiments, a pulse oximeter 106 is connected to data port 40. In some embodiments, the control assembly 12 further comprises a power switch 164 and a speaker 166. Within the control assembly 12 resides a power storage unit 66 and a circuit board 68. In some embodiments, the control assembly 12 comprises a sensor 58, for measuring conditions within the first housing 20, and an ambient air injector 64, having an air injection port 70. In some embodiments, the control assembly 12 further has a hole 78 to allow air to enter an interior space of the control assembly 12 and be injected into the cartridge assembly 14 via ambient air injector 64. In some embodiments, the control assembly 12 comprises a solenoid valve 87 in lieu of or in addition to the ambient air injector 64.

In some embodiments, the cartridge assembly 14 has an RFID chip 76 which communicates with a chip reader 75. The chip reader 75 can be used to read, write, and/or rewrite information on the RFID chip 76, depending upon the desired configuration. In sat least some embodiments, the RFID chip 76 and chip reader 75 employ near filed communication.

In some embodiments, the control assembly 12 comprises a gas analyzer 79 and the cartridge assembly 14 has a photoluminescent gas sensor 81. The photoluminescent gas sensor 81 can sample the gas within the cartridge assembly 14 and the gas analyzer 79 can analyze the results via a window 83, for example. Photoluminescent gas sensors may be particularly useful for use with rebreather style breathing apparatus 10 because such rebreathers tend to become filled with hot, moist air. As shown in FIG. 13, for example, the gas analyzer 79 is separated from the hot, moist air.

In some embodiments, the breathing apparatus 10 includes one or more ambient air pumps which can be used alone or in combination with normally open ambient air solenoids.

In some embodiments, the breathing apparatus 10 has a network connection, for example via WiFi or Ethernet port. Via the network connection, data can be reported to a prescribing physician, for example where the breathing apparatus 10 is used at home. The network connection can also be utilized for software upgrades, ordering replacement cartridge assemblies 14, downloading patient information, etc. In some embodiments, the breathing apparatus 10 is lightweight and portable.

A description of some embodiments of the breathing apparatus and method are contained in one or more of the following numbered statements:

Statement 1: A breathing apparatus comprising:

a breathing mask;

a hose attached to the breathing mask and defining an expiratory flow passage through which expiratory gas from the breathing mask flows, the hose further attached to a first port, the first port defining an inlet into a first housing such that the flow passage is in fluid communication with the first port;

the first port comprising a first directional valve for one-directional flow of the expiratory gas through the first port into the first housing;

the first housing having a first chamber and a second chamber, the second chamber separated from the first chamber by a barrier, the barrier comprising a carbon dioxide scrubber through which the expiratory gas flows after entering the first chamber;

an expandable reservoir in fluid communication with at least one of the first chamber and second chamber;

the barrier separating the first and second chambers having a second port, the second port comprising a second directional valve for one-directional flow of gas out of the second chamber; and wherein the second port communicates with the breathing mask via an inspiratory flow passage.

Statement 2. The breathing apparatus of statement 1, wherein at least a portion of the barrier separating the first and second chambers is non-permeable.
Statement 3. The breathing apparatus of statement 1, wherein the hose further defines the inspiratory flow passage.
Statement 4. The breathing apparatus of statement 3, wherein the inspiratory flow passage and expiratory flow passage are coaxial.
Statement 5. The breathing apparatus of statement 1, wherein the carbon dioxide scrubber includes a scrubbing media consisting of lithium chloride, lithium hydroxide, soda lime, calcium hydroxide, and combinations thereof.
Statement 6. The breathing apparatus of statement 1 further comprising an ambient air valve configured to selectively permit entry of ambient air into at least one of the first and second chambers;
Statement 7. The breathing apparatus of statement 6, wherein the ambient air valve comprises a normally open solenoid.
Statement 8. The breathing apparatus of statement 1 further comprising an ambient air pump configured to selectively provide ambient air into at least one of the first and second chambers.
Statement 9. The breathing apparatus of statement 1, wherein first housing comprises a disposable cartridge.
Statement 10. The breathing apparatus of statement 9, wherein the disposable cartridge comprises a near field communications device.
Statement 11. The breathing apparatus of statement 1 further comprising a barrier bypass which is configured to selectively permit gas to bypass the carbon dioxide scrubber.
Statement 12. The breathing apparatus of statement 1 further comprising a carbon dioxide sensor and an oxygen sensor.
Statement 13. The breathing apparatus of statement 12, wherein the carbon dioxide sensor and oxygen sensor are configured to measure the oxygen and carbon dioxide levels within the second chamber.
Statement 14. The breathing apparatus of statement 12, wherein the oxygen sensor comprises an optical sensor.
Statement 15. The breathing apparatus of statement 1 further comprising a spirometer.
Statement 16. The breathing apparatus of statement 1 further comprising a pulse oximeter.
Statement 17. The breathing apparatus of statement 1 further comprising a heart rate monitor.
Statement 18. The breathing apparatus of statement 1, wherein the breathing mask further comprises an accelerometer or position sensor.
Statement 19. The breathing apparatus of statement 1 further comprising a ventilation rate monitor.
Statement 20. The breathing apparatus of statement 1 further comprising a control apparatus.
Statement 21. The breathing apparatus of statement 20, wherein the control apparatus comprises an anti-microbial lining on at least a portion thereof.
Statement 22. A method of providing hypoxic therapy to a patient comprising:

providing a breathing apparatus having:
a breathing mask;
a port;
a control assembly; and
a hose attached to the breathing mask and port;

providing a gaseous mixture to the patient with the breathing apparatus;

monitoring the gaseous mixture provided to the patient with the breathing apparatus;

measuring the patient's oxygen saturation level;

measuring the rate of change of the patient's oxygen saturation level; and adjusting the contents of the gaseous mixture provided to the patient with the breathing apparatus.

Statement 23. The method of statement 22, wherein the breathing apparatus further comprises a near field communications device attached to at least one of the breathing mask and hose.
Statement 24. The method of statement 23, wherein the near field communications device utilizes RFID.
Statement 25. The method of statement 22 further comprising calculating the hypoxic training index with the breathing assembly.
Statement 26. The method of statement 22, wherein the step of adjusting the contents of the gaseous mixture comprises adjusting the oxygen content.
Statement 27. The method of statement 22, wherein the step of adjusting the contents of the gaseous mixture comprises adjusting the carbon dioxide content.
Statement 28. The method of statement 22, wherein the breathing apparatus comprises a pressure swing adsorption device.
Statement 29. The method of statement 22, wherein the breathing apparatus comprises a nitrogen enrichment device.
Statement 30. The method of statement 22, wherein the breathing apparatus comprises a hollow fiber membrane device.
Statement 31. The method of statement 22, wherein the breathing apparatus comprises a rebreathing device.
Statement 32. A method of administering hypoxic therapy comprising:

providing a mixture of hypoxic gas to a patient;

measuring the patient's physiological response to the mixture of hypoxic gas, including the rate at which the patient's arterial oxygen saturation is changing; and

adjusting the mixture of hypoxic gas provided to the patient in real time based on the patient's physiological response.

Statement 33. The method of statement 32, wherein the step of adjusting the mixture of hypoxic gas further comprises adjusting the mixture of hypoxic gas provided to the patient in real time based on the progression in administering a predetermined hypoxic dosage.
Statement 34. The method of statement 32 further comprising providing normoxic gas to the patient based on the patient's physiological response.
Statement 35. The method of statement 32, wherein the step of adjusting the mixture of hypoxic gas further comprises introducing ambient air into the mixture.
Statement 36. The method of statement 32, wherein the step of adjusting the mixture of hypoxic gas further comprises adjusting the oxygen concentration of the mixture.
Statement 37. The method of statement 32 further comprising predicting a physiological response from the patient.

The above disclosure is intended to be illustrative and not exhaustive. This description will suggest many variations and alternatives to one of ordinary skill in this field of art. All these alternatives and variations are intended to be included within the scope of the claims where the term “comprising” means “including, but not limited to.” Those familiar with the art may recognize other equivalents to the specific embodiments described herein which equivalents are also intended to be encompassed by the claims.

Further, the particular features presented in the dependent claims can be combined with each other in other manners within the scope of the invention such that the invention should be recognized as also specifically directed to other embodiments having any other possible combination of the features of the dependent claims. For instance, for purposes of claim publication, any dependent claim which follows should be taken as alternatively written in a multiple dependent form from all prior claims which possess all antecedents referenced in such dependent claim if such multiple dependent format is an accepted format within the jurisdiction (e.g. each claim depending directly from claim 1 should be alternatively taken as depending from all previous claims). In jurisdictions where multiple dependent claim formats are restricted, the following dependent claims should each be also taken as alternatively written in each singly dependent claim format which creates a dependency from a prior antecedent-possessing claim other than the specific claim listed in such dependent claim below.

This completes the description of the preferred and alternate embodiments of the invention. Those skilled in the art may recognize other equivalents to the specific embodiment described herein which equivalents are intended to be encompassed by the claims attached hereto.

What is claimed is:

1. A breathing apparatus comprising:
a breathing mask;
a hose attached to the breathing mask and defining an expiratory flow passage through which expiratory gas from the breathing mask flows, the hose further attached to a first port, the first port defining an inlet into a first housing such that the flow passage is in fluid communication with the first port;
the first port comprising a first directional valve for one-directional flow of the expiratory gas through the first port into the first housing;
the first housing having a first chamber and a second chamber, the second chamber separated from the first chamber by a barrier, the barrier comprising a carbon dioxide scrubber through which the expiratory gas flows after entering the first chamber;
an expandable reservoir in fluid communication with at least one of the first chamber and second chamber;
the barrier separating the first and second chambers having a second port, the second port comprising a second directional valve for one-directional flow of gas out of the second chamber; and
wherein the second port communicates with the breathing mask via an inspiratory flow passage.

2. The breathing apparatus of claim 1, wherein at least a portion of the barrier separating the first and second chambers is non-permeable.

3. The breathing apparatus of claim 1, wherein the hose further defines the inspiratory flow passage.

4. The breathing apparatus of claim 3, wherein the inspiratory flow passage and expiratory flow passage are coaxial.

5. The breathing apparatus of claim 1, wherein the carbon dioxide scrubber includes a scrubbing media consisting of lithium chloride, lithium hydroxide, soda lime, calcium hydroxide, and combinations thereof.

6. The breathing apparatus of claim 1 further comprising an ambient air valve configured to selectively permit entry of ambient air into at least one of the first and second chambers;

7. The breathing apparatus of claim 6, wherein the ambient air valve comprises a normally open solenoid.

8. The breathing apparatus of claim 1 further comprising an ambient air pump configured to selectively provide ambient air into at least one of the first and second chambers.

9. The breathing apparatus of claim 1, wherein first housing comprises a disposable cartridge.

10. The breathing apparatus of claim 9, wherein the disposable cartridge comprises a near field communications device.

11. The breathing apparatus of claim 1 further comprising a barrier bypass which is configured to selectively permit gas to bypass the carbon dioxide scrubber.

12. The breathing apparatus of claim 1 further comprising a carbon dioxide sensor and an oxygen sensor.

13. The breathing apparatus of claim 12, wherein the carbon dioxide sensor and oxygen sensor are configured to measure the oxygen and carbon dioxide levels within the second chamber.

14. The breathing apparatus of claim 12, wherein the oxygen sensor comprises an optical sensor.

15. The breathing apparatus of claim 1 further comprising a spirometer.

16. The breathing apparatus of claim 1 further comprising a pulse oximeter.

17. The breathing apparatus of claim 1 further comprising a heart rate monitor.

18. The breathing apparatus of claim 1, wherein the breathing mask further comprises an accelerometer or position sensor.

19. The breathing apparatus of claim 1 further comprising a ventilation rate monitor.

20. The breathing apparatus of claim 1 further comprising a control apparatus.

21. The breathing apparatus of claim 20, wherein the control apparatus comprises an anti-microbial lining on at least a portion thereof.

22. A method of providing hypoxic therapy to a patient comprising:
providing a breathing apparatus having:
a breathing mask;
a port;
a control assembly; and
a hose attached to the breathing mask and port;
providing a gaseous mixture to the patient with the breathing apparatus;
monitoring the gaseous mixture provided to the patient with the breathing apparatus;
measuring the patient's oxygen saturation level;
measuring the rate of change of the patient's oxygen saturation level; and
adjusting the contents of the gaseous mixture provided to the patient with the breathing apparatus.

23. The method of claim 22, wherein the breathing apparatus further comprises a near field communications device attached to at least one of the breathing mask and hose.

24. The method of claim 23, wherein the near field communications device utilizes RFID.

25. The method of claim 22 further comprising calculating the hypoxic training index with the breathing assembly.

26. The method of claim 22, wherein the step of adjusting the contents of the gaseous mixture comprises adjusting the oxygen content.

27. The method of claim 22, wherein the step of adjusting the contents of the gaseous mixture comprises adjusting the carbon dioxide content.

28. The method of claim 22, wherein the breathing apparatus comprises a pressure swing adsorption device.

29. The method of claim 22, wherein the breathing apparatus comprises a nitrogen enrichment device.

30. The method of claim 22, wherein the breathing apparatus comprises a hollow fiber membrane device.

31. The method of claim 22, wherein the breathing apparatus comprises a rebreathing device.

32. A method of administering hypoxic therapy comprising:
providing a mixture of hypoxic gas to a patient;
measuring the patient's physiological response to the mixture of hypoxic gas, including the rate at which the patient's arterial oxygen saturation is changing; and
adjusting the mixture of hypoxic gas provided to the patient in real time based on the patient's physiological response.

33. The method of claim 32, wherein the step of adjusting the mixture of hypoxic gas further comprises adjusting the mixture of hypoxic gas provided to the patient in real time based on the progression in administering a predetermined hypoxic dosage.

34. The method of claim 32 further comprising providing normoxic gas to the patient based on the patient's physiological response.

35. The method of claim 32, wherein the step of adjusting the mixture of hypoxic gas further comprises introducing ambient air into the mixture.

36. The method of claim 32, wherein the step of adjusting the mixture of hypoxic gas further comprises adjusting the oxygen concentration of the mixture.

37. The method of claim 32 further comprising predicting a physiological response from the patient.
